(19) United States
(12) Patent Application Publication
(10) Pub. No.: US 2026/0335072 A1
(43) Pub. Date: Oct. 29, 2026

(54) SIZE-ADJUSTABLE HEART VALVE CONDUIT

(71) Applicant: Autus Valve Technologies, Inc., Boston, MA (US)

(72) Inventor: Sophie-Charlotte Hofferberth, Boston, MA (US)

(73) Assignee: Autus Valve Technologies, Inc., Boston, MA (US)

(21) Appl. No.: 19/187,352

(22) Filed: Apr. 23, 2025

(51) Int. Cl. A61F 2/24

(52) U.S. Cl. CPC A61F 2/2418; A61F 2250/0039

(57) ABSTRACT

A geometrically accommodating valve in conjunction with an expandable conduit is provided for use in heart or other valve replacement treatment. The geometrically accommodating valve is fixed to the conduit in a manner to allow for radial expansion of the conduit and valve to adapt to the patient over time. The conduit containing the geometrically accommodating valve can include preformed sinuses to optimize flow characteristics through the valve and behind the valve leaflets. In particular, when the conduit has an unchanging circumference along its length, blood can become trapped in small cavities behind valve leaflets after flowing through the valve. Sinuses behind the valve leaflets allow blood flow behind the valve leaflets and prevent blood from being trapped and stagnant behind the valve leaflets. Sinuses result in an expanded circumference of the conduit in the area around the valve.

### TECHNICAL FIELD

This disclosure relates generally to valve replacement devices, and more specifically, to human valve replacement devices and valved conduits.

### BACKGROUND

The human heart includes a series of valves that work to ensure that blood flows correctly through the chambers of the heart. Birth defects, trauma, or other pathologies can negatively impact the function of a person's native heart valves. Prosthetic heart valves have been developed to either supplement or entirely replace defective native heart valves.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIGS. 1A-1C show various views of a geometrically accommodating valve in conjunction with a conduit for use in heart or other valve replacement treatment, in accordance with various embodiments.

FIG. 2 shows a geometrically accommodating valve, in accordance with various embodiments.

FIG. 3 shows device including a geometrically accommodating valve coupled to a conduit, in accordance with various embodiments.

FIG. 4 illustrates an example of a device including a conduit, a valve, and sinuses attached to a heart, replacing the pulmonary artery, in accordance with various embodiments.

FIG. 5A illustrates an expandable conduit coupled to a valve, according to various embodiments.

FIG. 5B illustrates a balloon catheter, which can be used to expand the perimeter of the conduit and/or the perimeter of the valve (and/or valve frame), in accordance with various embodiments.

FIGS. 6A-6D illustrate examples of various conduit shapes, in accordance with various embodiments.

FIG. 7 illustrates a conduit including a valve attached to a heart, replacing the pulmonary artery, in accordance with various embodiments.

FIG. 8 illustrates stress-strain comparisons on a conduit for difference types of conduit materials, in accordance with various embodiments.

FIGS. 9A-9B illustrate a support structure for a conduit, according to various embodiments.

FIG. 10 illustrates an example support structure, in accordance with various embodiments.

FIG. 11 illustrates three examples of valve placement and orientation within a conduit with respect to the heart for a pulmonary artery, in accordance with various embodiments.

FIG. 12 shows a device including a geometrically accommodating valve having a sinus coupled to a conduit, in accordance with various embodiments.

FIGS. 13A-13C illustrate various sinus shapes, in accordance with various embodiments.

FIG. 14 shows various views of sinuses 1425 with respect to a conduit 1420, in accordance with various embodiments.

FIG. 15A illustrates blood flow in the sinuses when the valve is open, in accordance with various embodiments.

FIG. 15B illustrates blood flow in the sinuses when the valve is closed, in accordance with various embodiments.

FIGS. 16A and 16B illustrate valve and conduit expansion methods, in accordance with various embodiments.

FIG. 17 illustrates a conduit and valve before radial expansion and after radial expansion, in accordance with various embodiments.

FIGS. 18A-18D illustrate various views of a non-expandable conduit including a non-expandable valve, in accordance with various embodiments.

FIGS. 19A-19D illustrate various views of a non-expandable valve, in accordance with various embodiments.

### DETAILED DESCRIPTION

### Overview

Systems, methods, and devices are provided for a geometrically accommodating valve in conjunction with an expandable conduit for use in heart or other valve replacement treatment. The geometrically accommodating valve is fixed to the conduit in a manner to allow for radial expansion of the conduit and valve to adapt to the patient over time. In particular, a pediatric patient may grow over time, and the patient's heart structures may grow with the rest of the patient's anatomy. As the patient grows, the geometrically accommodating valve can expand and adapt to the patient's anatomy. In some examples, the geometrically accommodating valve and conduit are designed to be expanded via a minimally invasive transcatheter balloon procedure. The balloon catheter can be used to cause radial expansion of the valve and conduit, and the conduit is designed to maintain its length despite the radial expansion. In particular, in various embodiments, the conduit is constrained by outside features to allow for radial expansion with minimal length contraction. In some examples, use of the geometrically accommodating valve for heart valve replacement can prevent one or more future valve replacement surgeries, when the purpose of the one or more future valve replacement surgeries is to replace a previously surgically placed heart valve with a larger heart valve due to patient growth.

In some implementations, the conduit containing the geometrically accommodating valve is designed to replace the pulmonary valve and at least a portion of the pulmonary artery, and in some examples, the conduit containing the valve is designed to replace all of the pulmonary valve and adjacent surrounding main pulmonary artery. In some implementations, the geometrically accommodating valve can be implanted in an existing pulmonary artery.

According to some implementations, the conduit containing the geometrically accommodating valve includes preformed sinuses to optimize flow characteristics through the valve and behind the valve leaflets. In particular, in some examples, when the conduit has an unchanging (or near uniform) circumference along its length, blood can become trapped in small cavities behind valve leaflets after flowing through the valve. Systems and methods provided herein include sinuses behind the valve leaflets, to allow blood flow behind the valve leaflets and prevent blood from being trapped and stagnant behind the valve leaflets. As discussed herein, sinuses result in an expanded circumference of the conduit in the area around the valve.

In some implementations the device, including the conduit and/or valve, can be used in an artery, and in some implementations, the device can be used elsewhere in the vascular system, for example, as a venous valve prosthesis. Additionally, while the conduit and/or valve can be used for a valved right ventricle to pulmonary artery connection, in other implementations, the conduit and/or valve can be used to replace other valves and/or in other areas of the vascular system. Additionally, in various implementations, the device, including the conduit and/or valve, may enable heart valve function in a diverse range of structural environments, across a range of structure sizes. The device can change in size and/or shape to accommodate varying structural environments.

According to some implementations, some embodiments of the conduit and valve replacement device can grow with the patient and maintain functionality across a range of sizes as the vasculature grows and the valve opening increases in size. Heart valve prosthetics that are designed for adults are intended to remain implanted for potentially up to decades at a time depending on the patient and the condition. In adults, vasculature lumen diameters and heart sizes do not generally change significantly from year to year. However, in children, vasculature and hearts grow significantly in size as the child matures. As a result, with static devices, children who have undergone a valve replacement at a young age have to undergo multiple procedures as they grow, to replace fixed size valves with suitably-sized valve replacement devices. Similarly, children who have undergone a conduit and valve replacement at a young age have to undergo multiple procedures as they grow, to replace fixed size conduits and valves with suitably-sized conduit and valve replacement devices. In some cases, adjusting or replacing a valve can require open-heart surgery. Thus, there is a need for a valve replacement device and for a conduit and valve replacement device that is effective over a range of sizes.

While embodiments described herein can be suitable for use in growing patients, it should be appreciated that the heart valve replacement devices described herein are not limited for use in growing patients. The devices may be used in non-growing applications as well.

In some embodiments, a first valve replacement device may be implanted in the first few years of a patient's life, and once the device reaches a full state of expansion, a second valve may be deployed inside the initial implanted device to enable the patient to avoid repeat open-heart procedures. In some implementations, when the valve reaches a full state of expansion, a new valve may be deployed inside the initial valve to enable the patient to avoid repeat open-heart procedures. In some implementations, the conduit can remain in place, implanted in the patient, the valve within the conduit can be removed, and the new valve can be placed inside the conduit. The new valve can be coupled to the conduit according to any selected attachment method or any combination of attachment methods. In some examples, the new valve can be stitched to the conduit and/or the new valve can be attached to the conduit with an adhesive. In various implementations, the new valve includes a sleeve, as described herein, and the new valve can be coupled to the conduit by attaching the sleeve portion of the new valve to the conduit.

In some implementations, as described herein, the device includes a valve and a conduit, and the valve functions as a valve within the conduit. The conduit can be a cylindrical tube connecting two heart structures, or some other expandable conduit system. For example, the valve portion of the device can be affixed to a cylindrical tube made of expandable synthetic material that acts as a right ventricle to pulmonary artery connection/conduit. In these embodiments, the valved conduit device may be expanded periodically via a transcatheter balloon dilation or other method to accommodate somatic growth of the patient and/or adjusted to match desired pressure and/or flow conditions.

Heart valve function can be characterized by different properties. One measure of heart valve function is regurgitant fraction, which is the amount of blood that leaks backwards through the valve against the intended direction of flow divided by the total amount of blood that flows through the heart valve in one stroke. A healthy, functional valve exhibits low amounts of regurgitation. In some examples, a regurgitant fraction of 0%-20% is desirable. Another measure of heart valve function is the amount of mean and peak transvalvular pressure gradient exhibited by the valve. This pressure metric is of interest because it quantifies valve function in allowing unimpeded forward flow. In some examples, a mean transvalvular pressure gradient of less than 40 mmHg is desirable.

In some implementations, the valve portion of the device includes one or more leaflets coupled to a frame. The leaflets surround an opening of the device through which blood can flow. The leaflets have an open configuration and a closed configuration. In the open configuration, the leaflets part and expose the opening of the device to permit blood flow through the opening. In the closed configuration, the leaflets coapt and obstruct the opening of the device to prevent backflow of blood. The leaflets move between the open and closed configurations based on the pressure differentials across the valve during the cardiac cycle. In some embodiments, the device includes two leaflets. However, other numbers of leaflets may be used, such as three, four, five or more leaflets. In some embodiments, the heart replacement device includes only one leaflet. The single leaflet may extend from one side of the frame to the other. In some embodiments, the leaflets are moon-shaped, e.g., ¾ to ½ moon shaped leaflets.

In some implementations, a frame portion of the valve may comprise a pair of semi-elliptical frame sections joined at a pair of commissures. The frame sections curve laterally outwards away from each other, forming a half-hourglass shape. As the surrounding vasculature widens, the two frame sections spread further apart at the open end, increasing the opening of the device. The two frame sections can spread apart at the open end to create an opening with a cross-sectional area similar to a top and bottom portions of the conduit. In some examples, the opening created by the frame sections can be increased when valve size and/or conduit size is increased. Each leaflet may be attached to the inner perimeter of each of the frame sections, mimicking the leaflet vessel wall attachment line of a native venous valve.

In some embodiments, the outer frame support may be a semi-rigid cylindrical mesh. The cylindrical mesh may have one or more segments along the length of the conduit. In some examples, the mesh may be shaped to accommodate two or more sinuses along the length of the conduit. For example, a cylindrical mesh can include two segments, and one or more sinuses may be positioned between the two segments. In some examples, the valve frame is attached to the inside of, and is kept stable by, the outer frame support. Due to the rigidity of the outer frame support, the inner surface of the outer frame support presses against the outer walls of the conduit portion of the device, keeping the lumen of the conduit open and maintaining the orientation and position of the device. The outer frame support can function similarly to a stent, allowing the device to be delivered via catheter delivery or direct surgical implantation. It should be appreciated however, that, in some embodiments, the conduit may be used on its own without the addition of the outer frame.

According to some implementations, the frame of the valve portion of the device maintains a constant perimeter length (e.g., the site of leaflet attachment is non-lengthening) throughout the growth process, i.e., the perimeter length does not stretch or elongate during growth. Instead, as the frame sections spread apart to accommodate growth, the height of the commissures reduces. In some embodiments of this non-lengthening design, accommodation of radial growth is achieved by balloon expanding a plastically-deformable material (e.g., steel, cobalt chromium, etc.). As a result, the leaflets attached to the frame sections do not become deformed (i.e., do not stretch, lengthen or unfold) as the valve opening expands. The extent of coaptation of the leaflets (i.e., length of leaflet material in contact with other valve leaflets in closed state) may decrease as the opening expands. In some embodiments, the frame sections of the frame are configured to maintain their shape as the device expands to accommodate growth, and thus the leaflets are not distended or stretched over the course of growth.

In some implementations, the frame and/or outer frame support of the valve portion of the device may be self-expanding or may expanded by other means, such as by balloon expansion.

In some implementations, the device, including the conduit and valve, may be delivered via minimally invasive means such as by a transcatheter approach or may be implanted via open-heart surgery.

In some implementations, the curve profile of the arcs of the frame sections can be obtained by projecting an elliptical quadrant on to a cylinder. In some embodiments, the cylinder can be representative of the shape of the inner wall of a vessel. The cylinder can have a radius equivalent to that of the valve held by the valve frame. In some examples, in an embodiment with 2 frame sections, the other frame section matches the projected curve profile but mirrored about the centerline of the cylinder. In various embodiments, other frame section shapes and curve profiles are contemplated. In various embodiments, other possible methods of defining the shape of the frame sections are contemplated and the current disclosure is not so limited. The frame sections may also have other shapes.

In some implementations, a valve portion of the device may include reinforcement features such as struts that connect the two frame sections of the valve frame. Such reinforcement struts may serve to maintain the shape of the valve frame during expansion. Some embodiments of the valve replacement device may include a top reinforcement strut that connects the two frame sections. The top reinforcement strut may be attached to the frame at or near the top of the pairs of commissures. The top reinforcement strut may have a length that is equal to or greater than the circumference of the frame opening at its maximum expansion diameter. The top reinforcement strut may form an annulus, or an ellipse, or may be asymmetrically shaped in its fully expanded state. In a non-expanded shape, the top reinforcement strut may have an undulating profile extending about a longitudinal axis of the valve replacement device such that the top reinforcement strut has a circular or elliptical or asymmetric profile when viewed from the top regardless of the state of the valve replacement device's expansion. In some embodiments, the top reinforcement strut may have undulations that gives the top reinforcement strut a smaller diameter before expansion of the frame but allowing the top reinforcement strut to expand with the frame. The top reinforcement strut may be comprised of a material with sufficient stiffness to provide reinforcing integrity to the frame as it expands, and with sufficient flexibility to allow the undulations to straighten out to permit the top reinforcement strut to expand.

In some implementations, the top reinforcement strut may have a variety of different geometries that may be varied according to the application of the valve replacement device. For example, the top reinforcing feature may be diamond shaped. In some examples, a diamond shaped top reinforcement strut may more readily allow the valve to be compressed over a catheter for percutaneous transvenous or transarterial valve deployment. In other examples, the top reinforcement feature may have any other selected shape. For instance, the top reinforcement feature can comprise multiple expandable segments connecting the frame sections instead of a single annulus. The segments can be telescoping segments or be otherwise folded or compressed to allow the feature to expand. The top reinforcement strut can also take on non-annular shapes as long as the feature can expand with the frame and fit within the implanted environment.

In some implementations, the valve portion of the device can include reinforcement features not attached to the commissures of the valve frame. For instance, some embodiments can have a lower reinforcement strut connecting the respective lower sides of the frame sections to each other. The lower reinforcement struts may also have an undulating or otherwise compressed or folded shape that allows the lower feature to expand with the frame. Similar to the top reinforcement strut, the lower feature may be comprised of a material with sufficient stiffness to give the valve frame additional structural strength, while being sufficiently flexible to allow the lower reinforcement strut to expand and/or straighten out or unfold.

In some implementations, the lower reinforcement strut may have a variety of different geometries that may be varied according to the application of the valve replacement device. For example, the top reinforcing feature may be diamond shaped to more readily allow the valve to be compressed over a catheter for percutaneous transvenous or transarterial valve deployment. In other examples, the lower reinforcement feature may have any other selected shape.

In some implementations, the top and lower reinforcement struts, or any other reinforcement strut, may also be tuned to alter or control the shape of the valve as the valve expands. The material properties, geometry, thickness, and/or other features of the reinforcement struts may be modified to achieve a specific opening geometry with expansion. The reinforcement struts may also differ in material, geometry, or thickness to achieve a specific opening geometry with expansion. One reinforcement strut may differ in material or thickness within said reinforcement strut to achieve a specific opening geometry with expansion. In some embodiments, the reinforcement struts can control the expansion of the frame to cause the opening to be elliptical in shape, asymmetrically shaped, circular in shape, or any other selected shape. For example, a reinforcement strut may be materially thicker on one side, or shorter on one side, or be made of a stiffer material on one side, to ensure that that side does not expand to the same degree as the other side of the frame

In some implementations, while top and lower reinforcement features are described, it should be understood that some embodiments can include one of or both of the top and lower reinforcement features. Additionally, some embodiments may include additional features that connect the frame sections and may be located between the top and lower reinforcement features.

In some implementations, the valve replacement device may include a plurality of holes spaced along the frame. The holes may act as anchor points for sutures to assist in the attachment of the leaflets to the frame. In some embodiments, the leaflets have additional protruding segments of material, and the additional protruding segments of material can be affixed to the frame via interspersing slots along the length of the leaflet attachment line. In some examples, the additional protruding segments of material can be mechanically fixed on the outside of the frame.

For purposes of explanation, specific numbers, materials and configurations are set forth in order to provide a thorough understanding of the illustrative implementations. However, it will be apparent to one skilled in the art that the present disclosure may be practiced without the specific details or/and that the present disclosure may be practiced with only some of the described aspects. In other instances, well known features are omitted or simplified in order not to obscure the illustrative implementations.

Further, references are made to the accompanying drawings that form a part hereof, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order from the described embodiment. Various additional operations may be performed or described operations may be omitted in additional embodiments.

For the purposes of the present disclosure, the phrase “A or B” or the phrase “A and/or B” means (A), (B), or (A and B). For the purposes of the present disclosure, the phrase “A, B, or C” or the phrase “A, B, and/or C” means (A), (B), (C), (A and B), (A and C), (B and C), or (A, B, and C). The term “between,” when used with reference to measurement ranges, is inclusive of the ends of the measurement ranges.

The description uses the phrases “in an embodiment” or “in embodiments,” which may each refer to one or more of the same or different embodiments. The terms “comprising,” “including,” “having,” and the like, as used with respect to embodiments of the present disclosure, are synonymous. The disclosure may use perspective-based descriptions such as “above,” “below,” “top,” “bottom,” and “side” to explain various features of the drawings, but these terms are simply for ease of discussion, and do not imply a desired or required orientation. The accompanying drawings are not necessarily drawn to scale. Unless otherwise specified, the use of the ordinal adjectives “first,” “second,” and “third,” etc., to describe a common object, merely indicates that different instances of like objects are being referred to and are not intended to imply that the objects so described must be in a given sequence, either temporally, spatially, in ranking or in any other manner.

In the following detailed description, various aspects of the illustrative implementations will be described using terms commonly employed by those skilled in the art to convey the substance of their work to others skilled in the art.

The terms “substantially,” “close,” “approximately,” “near,” and “about,” generally refer to being within +/−20% of a target value as described herein or as known in the art. Similarly, terms indicating orientation of various elements, e.g., “coplanar,” “perpendicular,” “orthogonal,” “parallel,” or any other angle between the elements, generally refer to being within +/−5-20% of a target value as described herein or as known in the art.

In addition, the terms “comprise,” “comprising,” “include,” “including,” “have,” “having” or any other variation thereof, are intended to cover a non-exclusive inclusion. For example, a method, process, device, neural network, or imaging system that comprises a list of elements is not necessarily limited to only those elements but may include other elements not expressly listed or inherent to such method, process, device, neural network, or imaging system. Also, the term “or” refers to an inclusive “or” and not to an exclusive “or.”

The systems, methods and devices of this disclosure each have several innovative aspects, no single one of which is solely responsible for all desirable attributes disclosed herein. Details of one or more implementations of the subject matter described in this specification are set forth in the description below and the accompanying drawings.

### Example Conduit and Valve

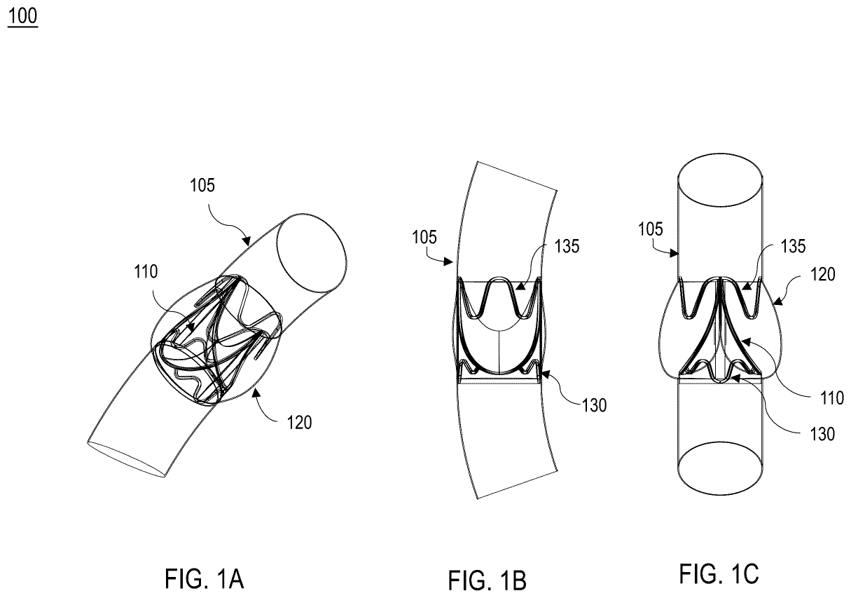

FIGS. 1A-1C show various views of a geometrically accommodating valve 110 in conjunction with a conduit 105 for use in heart or other valve replacement treatment, in accordance with various embodiments. The conduit can be configured to replace a portion of a vessel. In various examples, the conduit 105 is an expandable conduit, such that the perimeter of the conduit 105 can be expanded. In various examples, the diameter of a conduit can be expanded, the major axis diameter of a conduit can be expanded, the minor axis diameter of a conduit can be expanded, and/or another distance from one side of a conduit radially across to another side of the conduit can be expanded. Similarly, in various examples, the valve 110 is an expandable valve, such that the width of the valve 110 can be expanded. In various examples herein, a geometrically accommodating valve may also be referred to as an expandable valve.

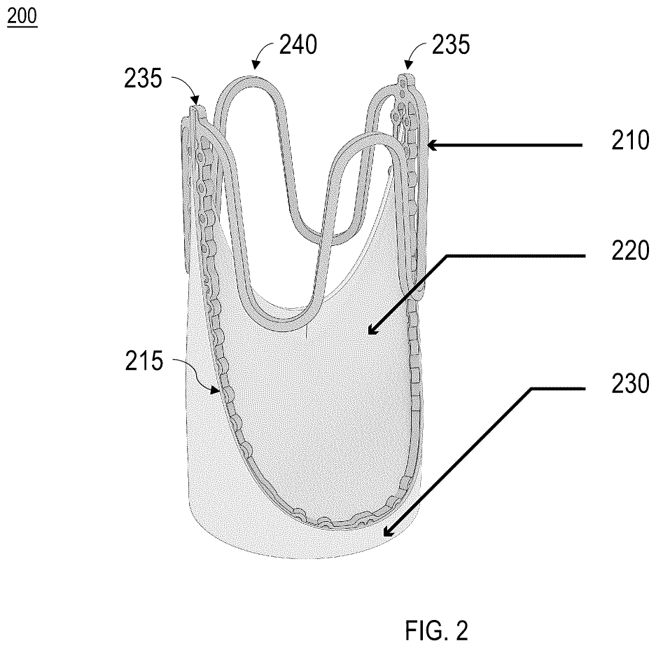

The valve 110 shown in FIGS. 1A-1C includes a frame including two frame sections surrounding two leaflets, an upper reinforcement strut 130, and a lower reinforcement strut 135. The frame sections may each be U-shaped arcs, and lining the arc of each frame section is a leaflet. The leaflets hang off of frame sections such that in the valve's closed configuration, the leaflets hang down and coapt, obstructing the valve 110 opening. As blood flows through the conduit 105 (flowing upwards from the bottom as viewed in FIGS. 1A-1C), the flow of blood pushes the leaflets apart, exposing the opening and placing the leaflets in an open configuration. As blood flow begins to slow, the leaflets once again collapse to the closed configuration, preventing regurgitation of blood. The valve 110 is described in greater detail with respect to FIG. 2. The upper 130 and lower 135 reinforcement struts are circular support features for the valve 110 and include undulations that allow for future expansion of the diameter of the upper 130 and lower 135 reinforcement struts and corresponding expansion of the valve 110. In particular, the undulations in the upper 130 and lower 135 reinforcement struts can become shallower to expand the valve 110, such that the largest circumference of the valve 110 is the circumference of the upper 130 and lower 135 reinforcement struts with the undulations straightened out.

The conduit 105 includes the valve 110 and an expandable sinus 120 behind the valve leaflets. FIGS. 1A and 1C show the expandable sinus 120 expanded behind the valve 110 leaflets. FIG. 1B shows a front-side view of the valve 110, such that the expandable sinuses 120 expand outward toward the front and back of the valve 110 (i.e., behind each valve leaflet), illustrating that the sideways expansion of the sinuses 120 is minimal. The expandable sinus 120 can be a bulbous area behind each valve 110 leaflet, which accommodates blood flow in the area behind the leaflets when the valve 110 is closed. In particular, the sinuses 120 optimize flow characteristics through the valve 110 and behind the valve leaflets. In particular, in some examples, when the conduit has an unchanging (or near uniform) circumference along its length, blood can become trapped in small cavities behind valve leaflets after flowing through the valve. Including sinuses 120 behind the valve 110 leaflets allows blood to flow behind the valve 110 leaflets and prevents blood from being trapped and stagnant behind the valve 110 leaflets. As shown in FIGS. 1A-1C, the sinuses 120 result in an expanded circumference of the conduit 105 in the area around the valve 110.

FIG. 2 shows a geometrically accommodating valve 200, in accordance with various embodiments. The valve 200 includes a frame 210, leaflets 220, and a sleeve 230. The frame 210 provides structure to the valve 200. The frame 210 is designed to allow for plastic deformation radially outward. In particular, the frame 210 includes two frame sections 215, and the two frame sections 215 converge at commissures 235, which are positioned at diametrically opposite points of the frame 210. The frame sections 215 may each be U-shaped arcs.

Lining the arc of each frame section 215 is a leaflet 220. The leaflets 220 are thin flexible membranes, and in some examples, the leaflets 220 are biomimetic leaflets. Each leaflet 220 may have a ¾ to ½ moon shape so that the concave outside matches the arc of the frame sections 215 that the leaflets 220 are attached to. The leaflets 220 may be sutured, adhered, or otherwise attached to the frame sections 215. A concave inside of the leaflets 220 may have a length of up to 4 times the diameter of valve 200 in baseline state/configuration, while the leaflet vertical height may be up to about 2.5 times the diameter of valve 220 in baseline state/configuration. The height of the center of the leaflet can range from 0.2 to 0.8 times the height of the leaflet at the commissures 235. As the frame 210 expands, the leaflet-commissure attachment angle increases, resulting in straightening of the leaflet's free edge. The excess leaflet height ensures that there is sufficient coaptation for the valve 200 to function effectively as a valve across the full range of the valve's expansion. The coaptation height can be up to three quarters of the opening diameter prior to valve frame expansion. While the depicted embodiments show equally sized and shaped leaflets 220 that are symmetric to one another, the valve leaflets 220 may be different in size and/or shape from one another. In some embodiments, one larger leaflet may cover the majority of the valve opening in the closed position, with one or more smaller leaflets forming a crescent-like shape in the closed position.

According to various implementations, the frame 210 may be made of nitinol, titanium, cobalt chromium alloy, stainless steel, a biodegradable polymer, a bioresorbable polymer, a synthetic material, platinum iridium, a magnesium or iron alloy, and/or any other suitable material.

The leaflets 220 open and close to enable unidirectional blood flow. The leaflets 220 hang off of frame sections 215 such that in the device's closed configuration, the leaflets 220 hang down and coapt, obstructing the valve 200 opening. As blood flows through the vessel (flowing upwards from the bottom as viewed in FIG. 2), the flow of blood pushes the leaflets 220 apart, exposing the opening and placing the leaflets 220 in an open configuration. As blood flow begins to slow, the leaflets 220 once again collapse to the closed configuration, preventing regurgitation of blood. The leaflets 220 have a geometry that allows the valve 200 to maintain coaptation (complete closure) across a wide range of diameters.

The size of the leaflets 220 can depend on the size of the valve, which can depend on the size of the person or animal in which the valve will be placed. In various examples, the free edge of the leaflet may have a length of between 2 to 6 times a circumference of the valve 200 or some other range. The leaflet vertical heights may be between 1.5 to 3.5 times the diameter of the valve 200 in the baseline configuration or 2.2 to 2.7 times the diameter of the valve 200 in an expanded configuration. The mid-height of the valve 200 may be from 0.1 to 1 times the height of the leaflet at the commissures. Other ranges are further contemplated. Additionally, while the depicted embodiments show designs utilizing two leaflets, designs using three or more leaflets with two or more frame sections are also contemplated. Designs using a single leaflet are also contemplated.

In various embodiments, the leaflets 220 can be made of a bioabsorbable polymer, a synthetic polymer, a tissue-engineered construct, a decellularized homologous tissue engineered leaflet, a thin film nitinol, an expanded PTFE membrane, a gluteraldehyde-treated bovine pericardium, a gluteraldehyde-treated porcine pericardium, a photo-oxidized bovine pericardium, a bovine jugular vein valve, or any other suitable composition or material. In some embodiments, the leaflets 220 are made of approximately 0.1 mm thickness expanded PTFE membrane having a Young's modulus of around 60 MPa at physiological loads. Young's modulus ranges from 30 MPa to 4 GPa, or 70 MPa to 4 GPa, or 100 MPa to 4 GPa, or 200 MPa to 4 GPa, or 500 MPa to 4 GPa for the leaflet material are also contemplated.

The frame 210 of the valve 200 includes an upper reinforcement strut 240. In some embodiments, the valve can also include a lower reinforcement strut, for example as shown in FIGS. 1A-1C. However, in other embodiments, the valve includes only one reinforcement strut. The upper reinforcement strut 240 is attached to the commissures 235 of the frame 210. The upper reinforcement strut 240 includes undulations. The undulations allow for potential expansion of the diameter of the upper reinforcement strut 240. In particular, the upper reinforcement strut 240 in FIG. 2 is shown in an unexpanded state, and as the frame 210 is expanded, the distance between the commissures 235 is increased, and the undulations begin to straighten out. The upper reinforcement strut 240 can be designed such that a total length of the upper reinforcement strut 240 may be equal to or larger than the largest possible circumference of the valve opening, such that the frame 210 can expand to the largest possible circumference of the valve opening and that the upper reinforcement strut 240 does not limit valve expansion or distort the geometry of the frame sections 215.

In some examples, the frame 210 includes features for affixing the sleeve 230 and leaflets 220 to the frame 210. The sleeve 230 wraps the frame 210 and forms a closed structure with the leaflets 220. In some examples, the sleeve 230 can be affixed to a conduit in which the valve 200 is positioned, and the sleeve 230 prevents flow around the valve 200 in the conduit. Additionally, the sleeve 230 can be the fixation point between the valve 200 and conduit, such that the valve 200 is coupled to the conduit via the sleeve 230.

While an annular upper reinforcement strut 240 with undulations is depicted in FIG. 2, it should be understood that any shape or arrangement that connects the commissures of the frame sections while allowing the frame to expand is also contemplated. For example, instead of an annular shape, the upper reinforcement strut 240 could be elliptical, diamond-shaped, or polygonal, or could comprise separate connecting portions instead of one continuous closed loop. The upper reinforcement strut 240 could also have a telescoping design, be made of an elastic material that can be reversibly deformed when the reinforcement strut 240 is expanded, or simply have more or fewer or larger or smaller undulations than depicted in the figures.

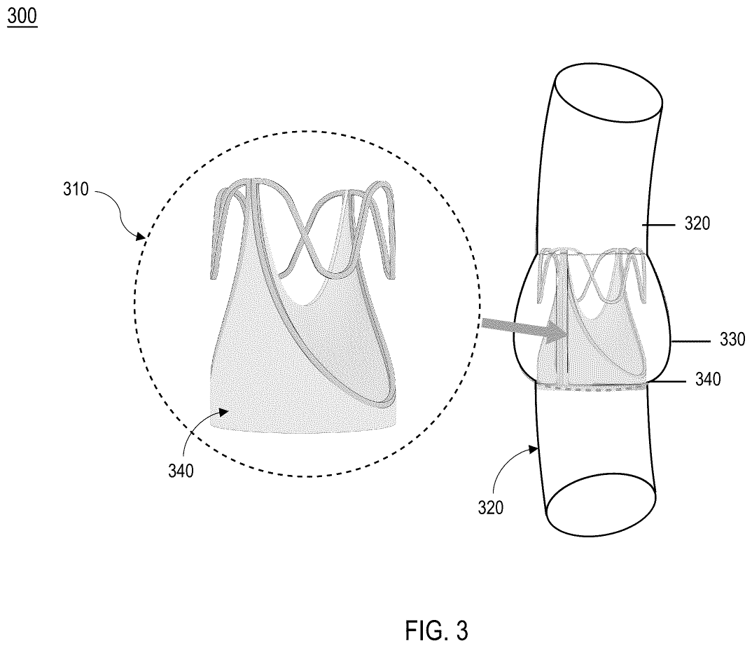

FIG. 3 shows device 300 including a geometrically accommodating valve 310 coupled to a conduit 320, in accordance with various embodiments. The conduit 320 is a flexible tube. In various embodiments, the conduit 320 is a cylindrical tube connecting two structures and/or connected to an artery or vein. In some examples, the conduit 320 replaces a portion of an artery, vein, or other tubular structure. The valve 310 is in the conduit 320, such that the conduit 320 holds the valve 310. In particular, the valve 310 is positioned in the conduit 320 and affixed to the conduit 320 around a lower portion of the sleeve 340 of the valve 310. The sleeve 340 is affixed to the conduit 320 such that the sleeve 340 prevents blood flow around the valve 310 in the conduit 320. In various embodiments, the sleeve 340 may be sutured, adhered, and/or otherwise attached to the conduit 320. In FIG. 3, blood flows up from the bottom of the figure, through a bottom portion of the conduit 320, and the sleeve 340 is affixed to the conduit 320 in such a manner to ensure that blood flowing up through the bottom portion of the conduit 320 flows through the valve 310.

In some embodiments, the conduit 320 can be plastically expanded to increase the diameter of the conduit 320. In some examples, the diameter of the conduit 320 and the diameter of the valve 310 can be expanded. For instance, a transcatheter balloon dilation procedure can be used to expand the conduit 320 and/or the valve 310. In some examples, the conduit 320 can be used to replace a portion of vascular, such as a portion of an artery or vein. As shown in FIG. 3, the conduit 320 can include sinuses 330 aligned with each leaflet of the valve 310. For instance, the sinuses 330 can be positioned behind each leaflet. As illustrated in FIG. 3, the circumference of the conduit 320 increases in the area where the sinuses 330 are positioned. The sinuses 330 can aid in flow dynamics, for example improving blood flow behind each leaflet of the valve. In other embodiments, the conduit 320 can be a non-expandable conduit.

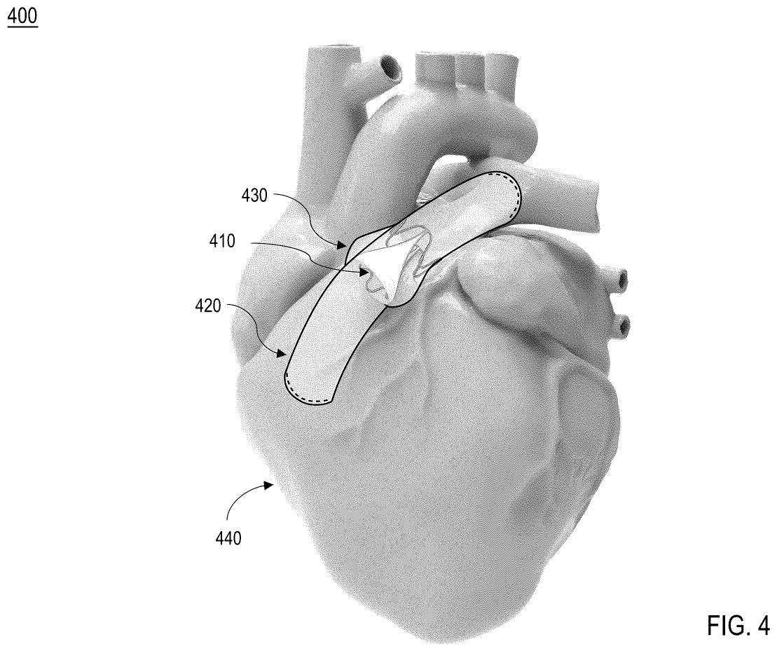

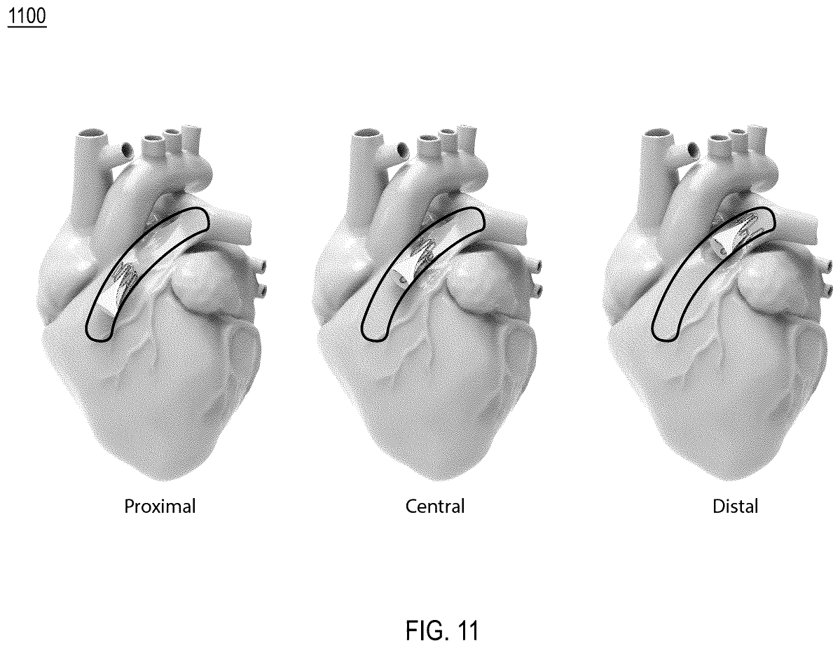

FIG. 4 illustrates an example of a device including a conduit 420, a valve 410, and sinuses 430 attached to a heart 440, replacing the pulmonary artery, in accordance with various embodiments. In some examples, device including the conduit 420 and valve 410 can be configured to address a malformation in the anatomy of the main pulmonary artery, and the device can be implanted in a patient to replace the entire main pulmonary artery, or a portion thereof. FIG. 4 illustrates a placement of the device including the conduit 420 and valve 410. In other embodiments, the device can be positioned differently. For example, FIG. 11, discussed below, illustrates some examples in which the valve is positioned differently within the conduit.

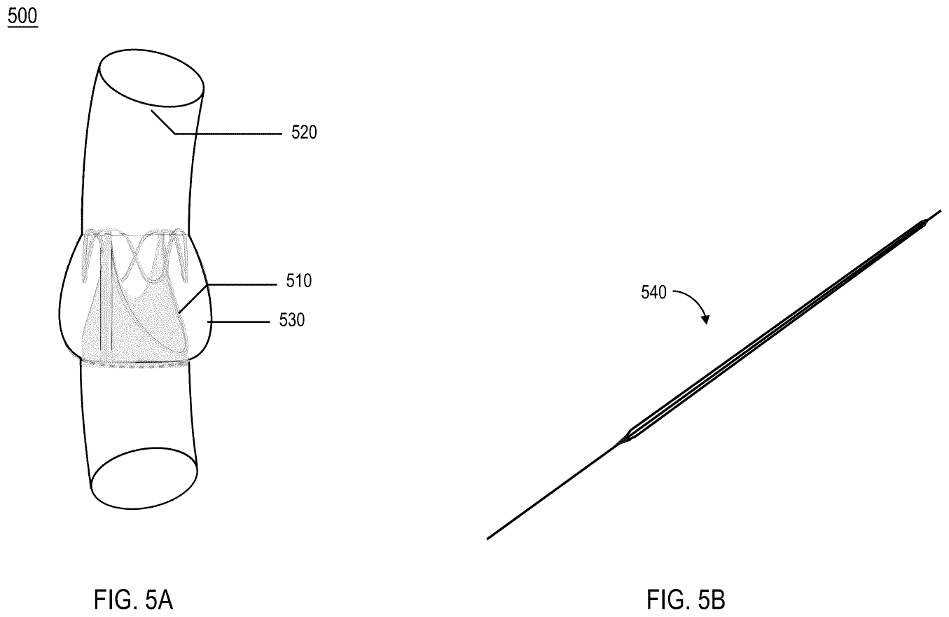

FIG. 5A illustrates an expandable conduit 520 coupled to a valve 510, according to various embodiments. The valve 510 can be substantially similar to any of the valves discussed herein, such as the valve 110, valve 200, and valve 310. The conduit 520 includes a sinus section 530, including sinuses positioned behind the leaflets of the valve 510. As shown in FIG. 5, the sinus section 530 of the conduit 520 has a larger diameter than the portions of the conduit that lie above and below the valve 510. In particular, the sinus section 530 has a sinus diameter that increases from the conduit diameter at the bottom of the valve 510 to a maximum diameter a portion of the way up the valve 510, and the sinus diameter decreases back to the conduit diameter around the top of the valve 510 frame. In some examples, the sinus section 530 expands when blood flows through the valve 510.

FIG. 5B illustrates a balloon catheter 540, which can be used to expand the perimeter of the conduit 520 and/or the perimeter of the valve 510 (and/or valve frame), in accordance with various embodiments. In particular, the balloon catheter 540 can be inserted into a patient's vasculature in a minimally invasive procedure and advanced to the heart to expand the perimeter of the conduit 520 and/or the perimeter of the valve 510 (and/or valve frame). In various embodiments, the balloon catheter 540 can be inflated after insertion to expand the perimeter of the balloon. Thus, the uninflated balloon catheter 540 can be inserted into a patient's vasculature, and then moved into position inside the conduit 520 and valve 510. Once in place inside the conduit 520 and/or valve 510, the balloon can be inflated to generate radial force, expanding the perimeter of the conduit 520 and/or valve 510. The balloon catheter can also expand the perimeter of the valve reinforcement struts. The balloon can subsequently be deflated for removal of the balloon catheter 540.

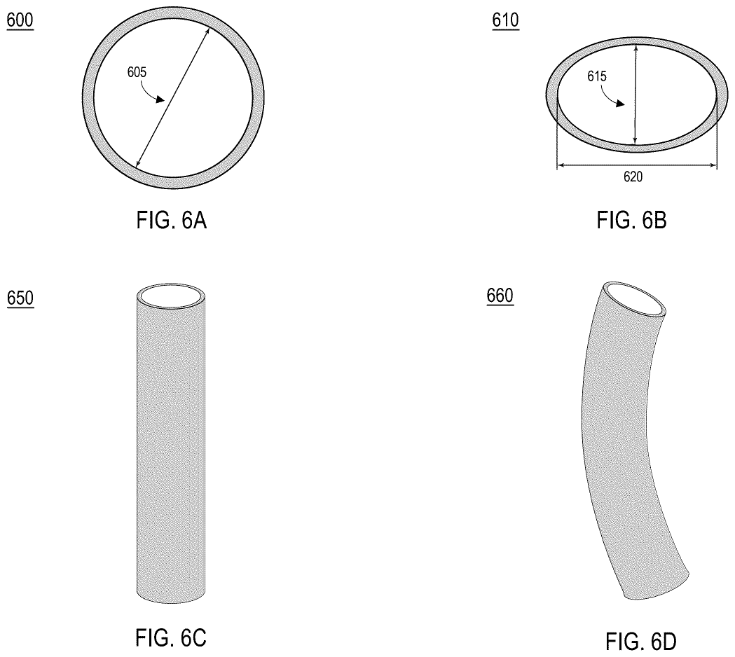

FIGS. 6A-6D illustrate examples of various conduit shapes, in accordance with various embodiments. In particular, FIGS. 6A and 6B illustrate examples of conduit cross-sections. FIG. 6A is a cross-sectional view 600 of a circular conduit having an interior diameter 605. In some examples, the interior diameter 605 can be between about 5 mm and about 50 mm, and in some examples, the interior diameter 605 can be between about 5 mm and about 30 mm. In some examples, the conduit is expandable, such that the interior diameter 605 can be expanded, for instance the interior diameter can be expanded from a first diameter to a second diameter, and wherein the interior diameter can be expanded from the first diameter by about 2 mm to by about 20 mm. In various implementations, the maximum size of the second diameter can vary depending on the device. For example, in various implementations, the interior diameter 605 can be expanded by about 2 mm, by about 3 mm, by about 4 mm, by about 5 mm, by about 6 mm, by about 7 mm, by about 8 mm, by about 9 mm, by about 10 mm, by about 15 mm, and/or by about 20 mm.

FIG. 6B is a cross-sectional view 610 of a conduit having an elliptical or oval-shaped cross-section. In particular, the conduit of FIG. 6B has a first interior width 615 across at its narrowest width and a second interior width 620 at its widest width. With reference to an ellipse shape, the conduit of FIG. 6B has a first interior width 615 at its minor axis and a second interior width 620 at its major axis. In some examples, the first interior width 615 is between about 3 mm and about 40 mm, and the second interior width 620 is between about 5 mm and about 75 mm. In some examples, the first interior width 615 is between about 5 mm and about 25 mm, and the second interior width 620 is between about 10 mm and about 50 mm. A valve can be configured to fit inside a conduit having an elliptical cross-sectional shape by matching the valve cross-sectional shape to the conduit cross-sectional shape.

FIGS. 6C and 6D illustrate two examples of conduit length shapes. FIG. 6C illustrates a side view of a conduit pre-formed to be straight. FIG. 6D illustrates a side view of a conduit pre-formed to be curved along its length. In various embodiments, a conduit can be formed to having a shape matching the portion of the vessel the conduit will be replacing when implanted in a patient.

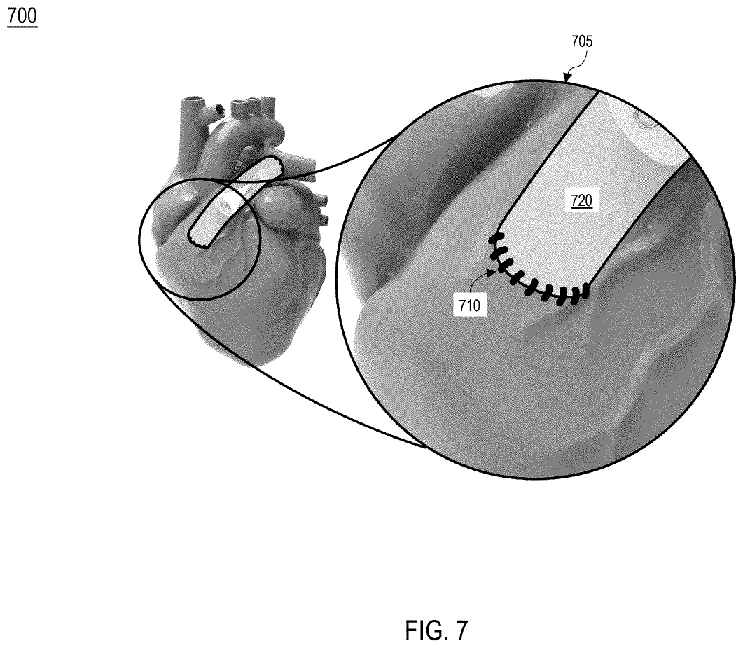

FIG. 7 illustrates a conduit 720 including a valve attached to a heart, replacing the pulmonary artery, in accordance with various embodiments. In particular, FIG. 7 includes an exploded view 705 of the attachment 710 between the heart and the proximal end of the conduit 720. As shown in FIG. 7, the proximal end of the conduit is sutured to the heart replacing a portion of the pulmonary artery at the exit from the heart. In some examples, the suture technique is such that the conduit can be expanded in subsequent procedures. For example, the suture technique can be a running suture technique, an interrupted horizontal mattress suture technique, and/or any other suture technique that allows for conduit expansion. That is, the attachment 710 site can also be expanded. In particular, the sutures do not prevent expansion of the conduit 720. In various embodiments, the conduit 720 is radially expandable, and the attachment 710 site is radially expandable to match the radius (or cross-sectional shape) of the conduit 720. The conduit 720 can expand radially while maintaining the conduit length, such that the distal attachment site at the distal end of the conduit is not moved closer or further from the proximal attachment 710 site. In some embodiments, the conduit 720 can expand radially and lengthwise, increasing both in diameter and in length. In various examples, both the proximal and distal ends of the conduit 720 are attached to the heart via sutures.

In various implementations, the conduit discussed herein can be made of a polymer with any of various expansion properties. In some examples, the conduit can be elastic and include a structure to maintain the conduit diameter. In some examples, the conduit can be plasticly deformed to retain an expanded form. In some implementations, the conduit can be made from expanded polytetrafluoroethylene, or a composite material containing expanded polytetrafluoroethylene.

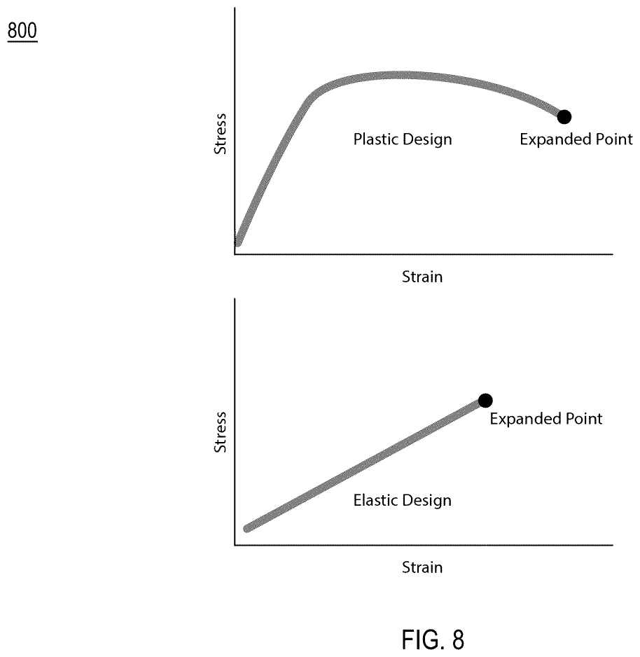

FIG. 8 illustrates stress-strain comparisons on a conduit for difference types of conduit materials, in accordance with various embodiments. The top graph illustrates the strain vs. stress on a conduit comprised of plastic material. In various examples, stress can be defined as the ratio of internal resistance developed by the conduit to resist deformation to the area of the cross-section, while strain is the measure of that deformation relative to the original dimensions of the material (i.e., the ratio of the change in the dimension to the original dimensions).

As shown in the top graph of FIG. 8, the strain and stress increase together to a selected point after which the stress on the material remains relatively constant even while the strain on the material increases. Thus, using a plastic material, the conduit can be expanded past the maximal stress point, to the plotted expanded point, further increasing strain on the material while not increasing stress on the material. Examples of plastic materials that can be used for the conduit material include polymers, such as a bioabsorbable polymer and/or a synthetic polymer, PTFE (polytetrafluoroethylene), and/or ePTFE (expanded polytetrafluoroethylene, which can be a microporous version of PTFE).

The bottom graph illustrates strain versus stress on a conduit comprised of an elastic material. Examples of plastic materials include materials that can elastically stretch and widen. As shown in the bottom graph, the strain and stress increase together linearly and consistently, such that as the strain increases, the stress also increases. Thus, when an elastic material is used, a support structure can be included to maintain conduit diameter. The support structure can be made from PTFE, ePTFE, and/or a polymer. Examples of support structures are described with respect to FIGS.

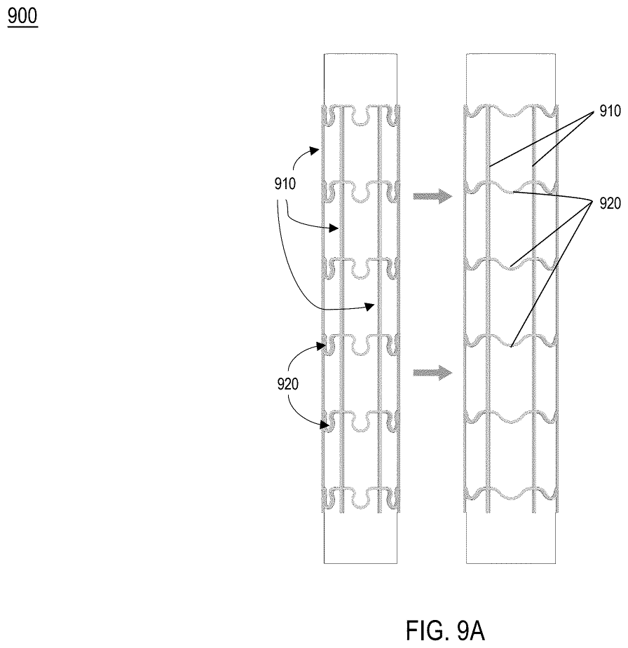

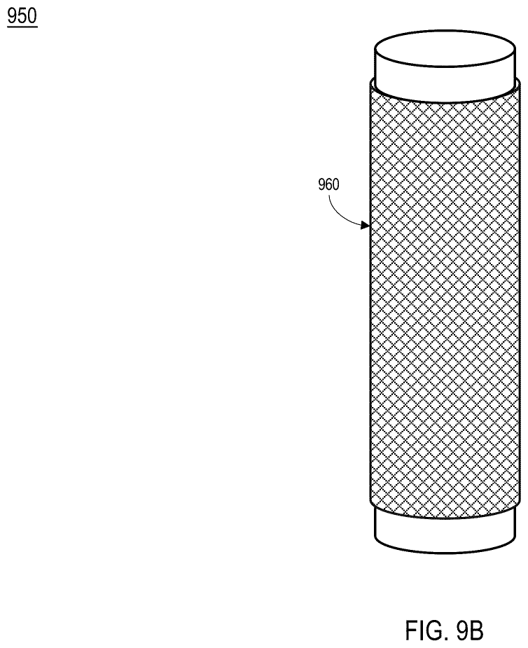

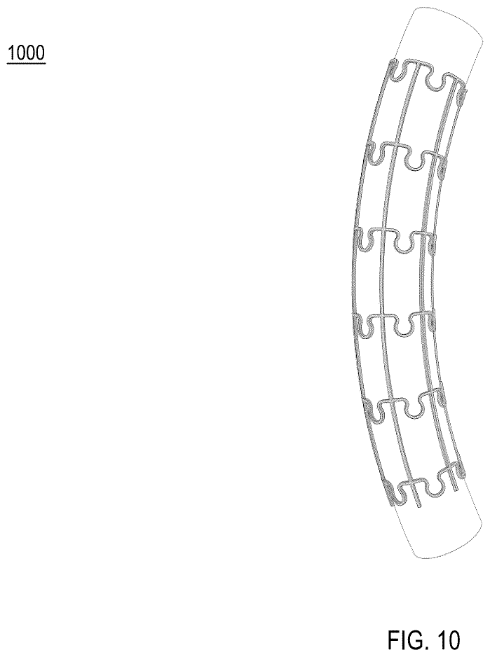

FIGS. 9A-9B and 10 illustrate support structures for a conduit, according to various embodiments. As shown in FIGS. 9A, 9B, and 10, the support structure is attached to the outside of the conduit such that the support structure does not come into contact with fluid flowing through the conduit. FIG. 9A illustrates a first example of a support structure for a conduit in an unexpanded state and in an expanded state, according to various embodiments. In particular, the left-hand side of FIG. 9A shows a conduit in an unexpanded shape. The structure can include multiple longitudinal rigid members 910 along its length and multiple flexible radially extendable expansion members 920 around its perimeter. The longitudinal members 910 can be attached to the radially extendable members 920. While in some instances a conduit may have a tendency to reduce in length as the material and valve are expanded, the conduits discussed herein are designed to maintain conduit length when conduit perimeter is expanded. In some examples, the rigid members 910 can help prevent any reduction in length of the conduit. In other examples, the conduit material is selected such that the conduit length remains consistent when the conduit cross-section is radially expanded.

In various embodiments, the additional support structure of FIGS. 9A, 9B, and 10 helps to maintain the conduit shape and protect the conduit from external compression forces. Additionally, according to various implementations, when the conduit is expanded, for example, using a balloon catheter, the support structure can help maintain the radial expansion of the conduit.

The support structure can be an outer frame that may be attached to the conduit at the intersection of the longitudinal members and the radial members. In various implementations, the support structure materials can include a first material for the longitudinal members and a second material for the radially extendable members. In some examples, the first and second materials are the same material. In some examples, the rigid longitudinal members are made from a non-expandable material. In some examples, the flexible radially extendable members are made from a ductile, balloon-expandable material. The materials can include metals and non-metals. In some examples, the materials can include stainless steel, cobalt-chromium, and/or titanium. In various examples, the structure can be made of metal, a polymer, and/or a combination of the material such as a metal and a polymer

FIG. 9B illustrates an example support structure 960 surrounding a conduit, in accordance with various embodiments. The support structure 960 can be a mesh support structure. The mesh may be a semi-rigid cylindrical mesh. The conduit is attached to the inside of the mesh support structure 960. Due to the rigidity of the support structure 960, the conduit can remain open despite outside pressure. Additionally, the mesh support structure 960 can be radially expanded while maintaining longitudinal rigidity (and thereby maintaining its length), similar to the support structure shown in FIG. 9A. The conduits discussed herein are designed to maintain conduit length when conduit perimeter is expanded. In some examples, the mesh support structure 960 can help prevent any reduction in length of the conduit with radial expansion. In some examples, the mesh support structure 960 can be made from materials such as stainless steel, cobalt-chromium, and/or titanium. In various examples, the structure can be made of metal, a polymer, and/or a combination of the material such as a metal and a polymer.

In various examples, the support structure can surround a conduit that includes one or more sinuses as described herein. In some examples, the support structure can surround the portion of the conduit proximal to the sinus and/or the portion distal to the sinus. In some examples, the support structure can surround the entire length of the conduit that includes the sinus. When the support structure surrounds the portion of the conduit that includes the sinus, the support structure in the area of the sinus can be different from the support structure in the areas proximal and/or distal to the sinus. For instance, the support structure in the area of the sinus can have a size and shape similar to the size and shape of the sinus.

FIG. 10 illustrates an example support structure 1000, in accordance with various embodiments. The support structure shown in FIG. 10 illustrates an example in which the structure can be formed to match the curve of the underlying conduit so that it fits the conduit shape while also providing support for the conduit. In various examples, the structure can be made of metal, a polymer, and/or a combination of the material such as a metal and a polymer.

In various embodiments, a support structure positioned around a conduit can protect the conduit against compressive forces, thereby helping preserve conduit shape. While some examples embodiments are shown herein, the support structure can have any selected geometry that serves the purpose of helping maintain conduit length while allowing for radial expansion of the conduit. In some examples, the support structure can have a continuous surface, a surface with open cells, a solid surface, and/or have any selected pattern or design. In some examples, the support structure can include an area with an enlarged perimeter to accommodate one or more sinuses. In some examples, the support structure can be segmented to include one or more support structure segments along a conduit length, and one or more support structure segments along a sinus, wherein the support structure segments along the sinus are shaped to accommodate a sinus shape. In some examples, the support structure can be segmented to include one or more support structure segments along a conduit length, and a support structure gap in a sinus area.

In various embodiments, the support structure can be attached to the conduit according to any selected attachment method and/or combination of attachment methods. In some examples, the conduit and the support structure are bonded together. In some examples, the conduit and support structure are attached using an adhesive. In some examples, the conduit and support structure are attached using sutures.

FIG. 11 illustrates three examples of valve placement and orientation within a conduit with respect to the heart for a pulmonary artery, in accordance with various embodiments. In particular, the valve can be situated linearly in any position along the conduit. The left heart illustration shows the valve positioned at a proximal portion of the conduit. The middle heart illustration shows the valve positioned close to the center of the conduit. The right heart illustration shows the valve positioned at a distal portion of the conduit. In some embodiments, it can be preferable to position the valve in the center of the conduit, in some embodiments, it can be preferable to position the valve at the proximal end of the conduit, and in some embodiments, it can be preferable to position the valve at the distal end of the conduit. In various embodiments, the surgeon may trim/shorten the length of the conduit proximal and/or distal to the valved portion of the conduit.

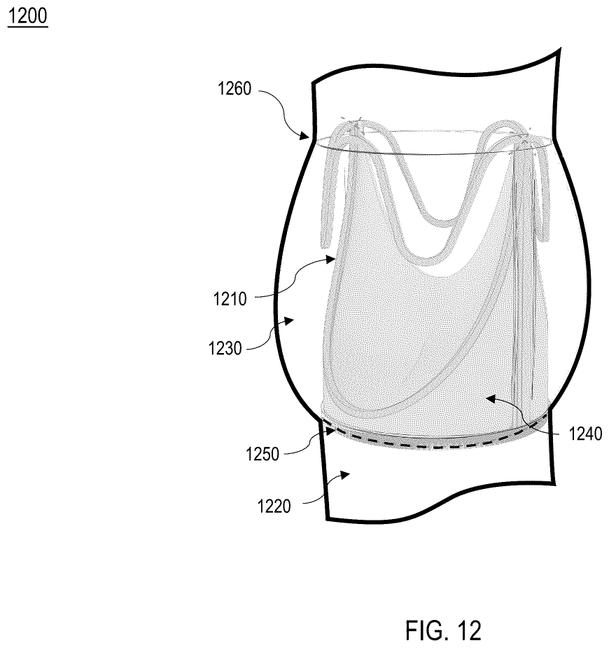

FIG. 12 shows a device 1200 including a geometrically accommodating valve 1210 having a sinus 1230 coupled to a conduit 1220, in accordance with various embodiments. The valve 1210 includes a sleeve 1240, and the proximal end of the valve 1210 can be attached to the conduit 1220 along a proximal end 1250 of the sleeve 1240. In FIG. 12, the attachment line at the proximal end 1250 of the sleeve 1240 is marked with a dashed line. In various examples, the sleeve 1240 of the valve 1210 can be fixed to the conduit 1220 using sutures, an adhesive, ultrasonic bonding, heat bonding and/or any other attachment method. In some embodiments, more than one attachment method can be used. The attachment between the conduit 1220 and sleeve 1240 is selected such that there are no openings or potential leakage and all blood flowing through the conduit 1220 flows toward the opening of the valve 1210.

In some implementations, the distal end of the valve 1210 remains unattached to the conduit 1220.

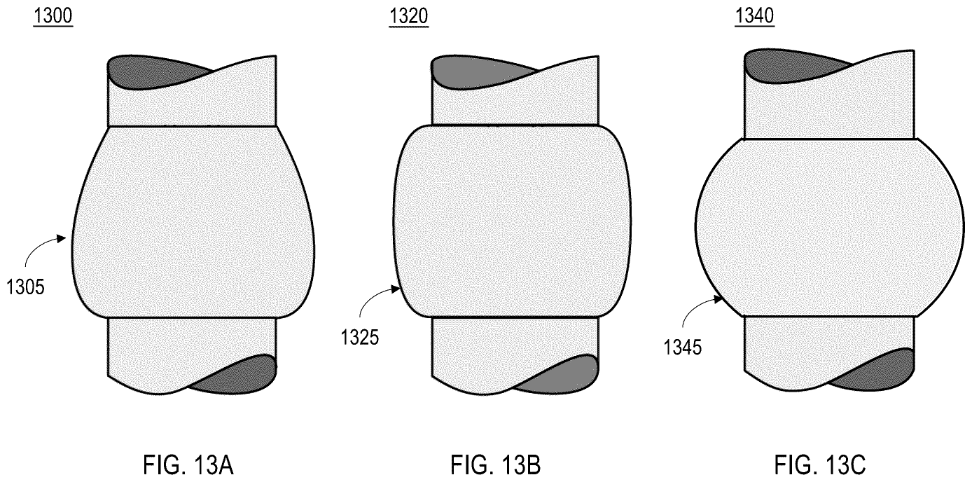

FIGS. 13A-13C illustrate various sinus shapes, in accordance with various embodiments. The sinuses are expanded spaces behind the valve leaflets. The expanded space of the sinuses facilitates vortex formation and blood flow reversal behind the valve leaflets, ensuring that blood that enters the space behind the leaflets does not become stagnant In particular, by providing additional space behind the leaflets, wash-out is facilitated. When the cavity behind the leaflet is narrow, flow stasis may occur, leading to potential blood clots or other issues.

In some implementations, the one or more sinuses are located behind the valve leaflets. In some examples, the area of the conduit in which the one or more sinuses are located can have a larger perimeter than the perimeter of the conduit above and below the valve. In some examples, the area of the conduit in which the one or more sinuses are located can have a variable perimeter that varies from proximal end of the sinus to a distal end of the sinus. The proximal end of the sinus can be located at a portion of the conduit that aligns with the proximal end of the valve. The distal end of the sinus can be located at a portion of the conduit that aligns with the distal end of the valve. The perimeter of the conduit at the area where the sinuses are located, and thus in the area of the valve, is larger than the perimeter of the conduit above and below the valve.

In some examples, the section of the conduit proximal to the sinus area (“the proximal conduit section”) and the section of the conduit distal to the sinus area (“the distal conduit area”) can have a diameter of between about 5 mm and about 50 mm, and thus, the proximal conduit section and the distal conduit section can have a corresponding perimeter of between about 30 mm and about 320 mm. In some examples, the area of the conduit in which the one or more sinus areas are located can have a perimeter that is between about 40 mm and about 600 mm.

FIG. 13A shows a conduit 1300 having one or more sinuses 1305 that have a teardrop shape that is weighted to the bottom. As shown in FIG. 13A, the perimeter of the conduit 1300 in the area of the one or more sinuses 1305 is larger than the perimeter of the conduit below and above the sinuses 1305. As shown in the teardrop shape of the sinuses 1305, the perimeter of the conduit 1300 at a proximal portion of the sinuses (shown as the bottom of the conduit in FIG. 13A) is larger than the perimeter of the conduit 1300 at a distal portion of the sinuses.

FIG. 13B shows a conduit 1320 having one or more sinuses 1325 that have a rounded and/or bulbous shape. As shown in FIG. 13B, the perimeter of the conduit 1320 in the area of the one or more sinuses 1325 is larger than the perimeter of the conduit below and above the sinuses 1325. As shown in the bulbous shape of the sinuses 1325, the perimeter of the conduit 1320 has a fairly symmetrical expansion in the area of the one or more sinuses 1325.

FIG. 13C shows a conduit 1340 having one or more sinuses 1345 that have a rounded and/or bulbous shape that is more spherical than the bulbous shape of the sinuses 1325 shown in FIG. 3B. As shown in FIG. 13C, the perimeter of the conduit 1340 in the area of the one or more sinuses 1345 is larger than the perimeter of the conduit below and above the sinuses 1345. As shown in the bulbous shape of the sinuses 1345, the perimeter of the conduit 1340 has a fairly symmetrical expansion in the area of the one or more sinuses 1345.

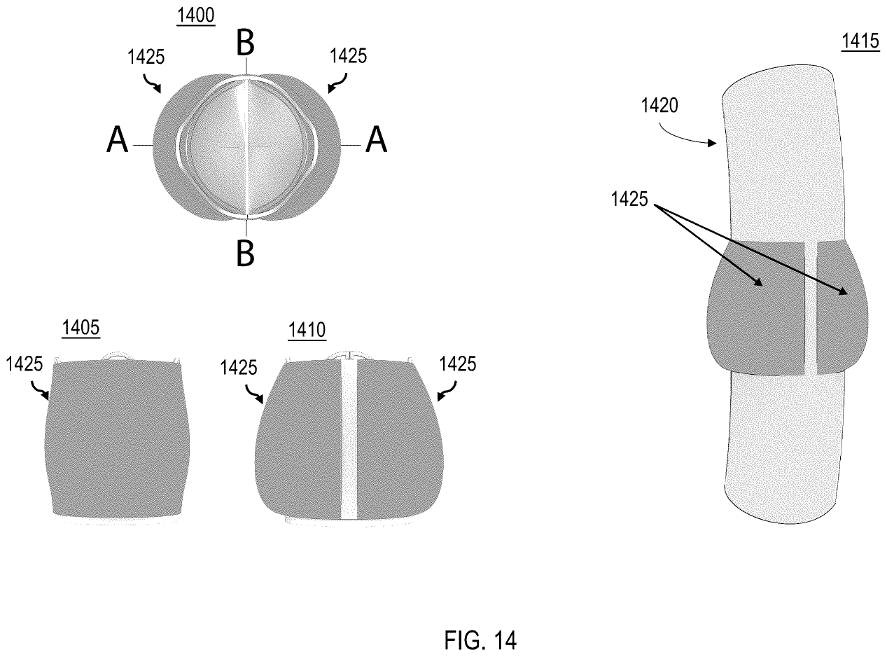

FIG. 14 shows various views of sinuses 1425 with respect to a conduit 1420, in accordance with various embodiments. In particular, FIG. 14 shows a top view 1400 of the sinuses 1425, a side first view 1405 of the sinuses 1425, and a rotated side view 1410 of the sinuses 1425, a side view 1415 including the conduit 1420 and the sinuses 1425. The sinuses 1425 are on both sides of the conduit 1420 in line with the valve. The top view 1400 shows a cross-section of the conduit 1420, with each sinus 1425 bulging out perpendicular to the leaflets (at point A), and the sinuses return to the conduit at the mid point (at point B) of the valve. Thus, as shown in the top view 1400, the sinuses 1425 are not circular in that the sinuses 1425 do not increase the perimeter of the conduit 1420 equally in all directions. Rather, the sinuses 1425 increase the perimeter of the conduit 1420 outward towards point A (behind the valve leaflets), thereby increasing the diameter of the conduit 1420 at points A, without increasing the diameter of the conduit 1420 at points B. In some examples, the sinuses 1425 can be preformed into the conduit 1420 material by plastic deformation of the material. In some examples, the sinuses 1425 can be made of another material and bonded to the conduit 1420. When sinuses 1425 are made of another material, holes in the conduit 1420 may be cut to allow for the insertion of the sinus portions. Additionally, when sinuses 1425 are made of another material, the sinuses can be bonded to the conduit 1420 via any bonding method, such as heat bonding, chemical bonding, ultrasonic bonding, and/or any other type of bonding.

In various embodiments, the sinus shape is configured to optimize blood flow within the sinuses when the valve is open and closed.

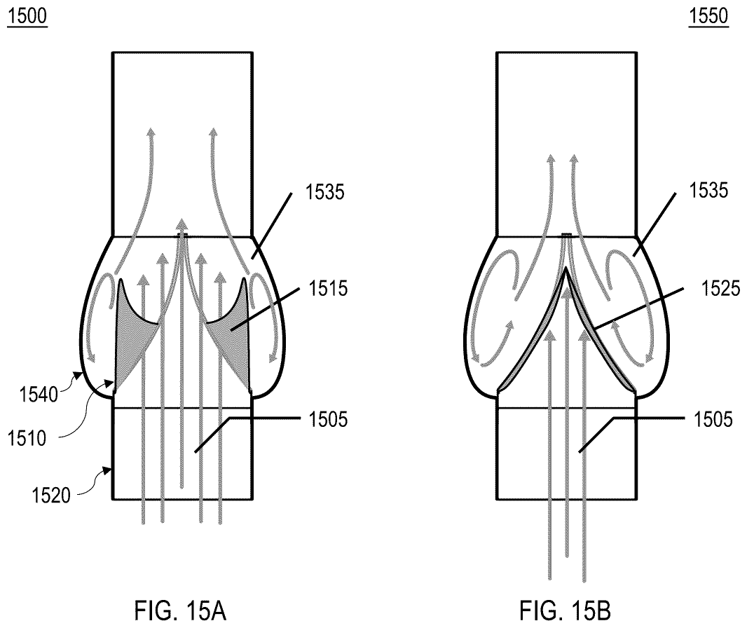

FIG. 15A illustrates blood flow in the sinuses when the valve is open, in accordance with various embodiments. FIG. 15B illustrates blood flow in the sinuses when the valve is closed, in accordance with various embodiments. FIGS. 15A and 15B each show a conduit 520, a valve 1510, and sinuses 1540. Blood is flowing from the proximal end of the conduit 1520 (at the bottom of the figures), through the valve 1510, toward the distal end of the conduit 1520. As discussed with respect to FIG. 13, the sinuses 1540 are a bulbous portion of the conduit 1520 in the region of the valve 1510. When the valve leaflets open and close, the presence of the sinuses 1540 improves blood flow in the region around the outer surface of the leaflets to expel any stagnate fluid.

FIG. 15A shows blood flow in the sinuses 1540 when the leaflets 1515 are open, in accordance with various embodiments. While most of the blood flows through the valve 1510 toward the distal end of the conduit 1520, a small amount of blood flows down into the sinuses 1540 as a result of vortice formation. In some examples, a pattern of vortex formation and flow reversal behind the leaflets into the valve base and/or sinuses reflects a biomimetic design of the valve, such that the valve design results in blood flow mimicking human venous valve flow. The blood flow in the sinuses 1540 is illustrated by the circular arrows behind the leaflets 1515. As shown by the arrows, blood that flows up through the valve 1510 can flow outward toward the outside surface of the leaflets, down toward the bottom of the sinuses, and then up along the inside edge of the sinuses and back up through the distal section of the conduit. While the desired direction of blood flow is through the distal section of the conduit, some blood can flow back behind the leaflets to washout the outflow surface of the valve, and the presence of the sinuses allows blood that flows behind the leaflets to circulate and exit through the distal section of the conduit rather than becoming stuck and or stagnant behind the leaflets.

FIG. 15B shows blood flow in the sinuses 1540 when the leaflets are in a closed position 1525. Blood flow through the valve is stopped when the valve 1510 is closed, and blood that is already on the distal side of the valve 1510 flows in a circular motion down into the sinuses 1540 and flows back up toward the distal end of the conduit 1520, as illustrated by the arrows behind in the sinuses 1540. Similar to the blood flow when the valve 1510 is open, as shown by the arrows, blood that has flowed up through the valve 1510 can flow outward toward the outside of the sinuses, down toward the bottom of the sinuses, and then up along the inside edge of the sinuses and back up through a distal section of the conduit.

As discussed above, many of the conduit and valve devices provided herein are expandable, such that the conduit and/or valve can be expanded to accommodate vasculature size of a patient. In some examples, the conduit and/or valve can be expanded after placement in a patient, and in some examples, the conduit and/or valve can be expanded multiple months and/or multiple years after placement of the conduit and valve. The conduit and/or valve can be expanded to adjust the flow to meet the patients needs as they grow and/or to accommodate other patient needs. In various implementations, the conduit and valve system can be expanded via a balloon catheter at after initial placement. In particular, one method of expansion is by use of a balloon catheter placed in the conduit and expanded. The expansion of the balloon causes the valve frame and the conduit to plasticly deform. When the balloon is removed, the valve and conduit maintain the new expanded size with minimal recoil. In some embodiments, the balloon catheter can expand the entire conduit and valve at once. In some embodiments, the balloon catheter can expand the conduit in segments advancing over the tube length.

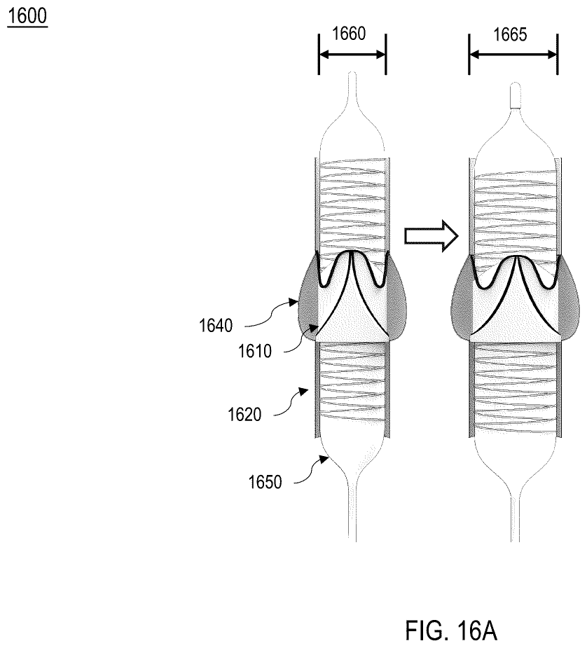

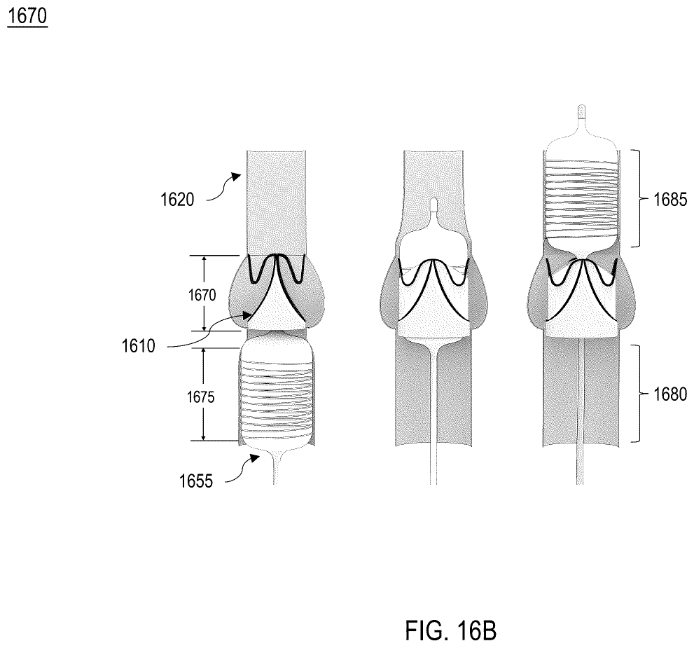

FIGS. 16A and 16B illustrate valve and conduit expansion methods, in accordance with various embodiments. FIG. 16A illustrates a balloon catheter 1650 expanding the entire conduit 1620 and valve 1640 at the same time. In particular, a single ballon 1650 is positioned through the conduit 1620 and valve 1610. The lefthand illustration shows the conduit 1620 and valve 1610 pre-expansion, having a first diameter 1660, with the balloon 1650 inside the conduit 1620 and valve 1610. After the balloon 1650 is in position, the balloon 1650 is further inflated to expand the conduit 1620 and valve 1610. The righthand illustration shows the conduit 1620 and valve 1610 post-expansion, having a second diameter 1665, with the inserted balloon further inflated to cause the expansion. Note that the undulations on the frame of the valve 1610 are shallower in the righthand figure as they have begun to straighten to allow for the expansion. The expansion shown in FIG. 16A is completed over the entire conduit 1620 and valve 1610 at the same time.

FIG. 16B illustrates a shorter balloon 1655 expanding the conduit 1620 in stages, in accordance with various embodiments. The balloon 1655 has a balloon length 1675 that is shorter than the conduit length proximal to the valve, allowing for expansion of the conduit 1620 in stages. In some examples, the length 1675 of the balloon is greater than the length 1670 of the valve 1610 to ensure even expansion of the valve 1610 in one step. The lefthand illustration shows the balloon 1655 inserted into a proximal section 1680 of the conduit 1620, and inflated to expand the proximal section 1680 of the conduit 1620. The center illustration shows the balloon 1655 inserted into the valve 1610, expanding the valve 1610 to substantially the same diameter as the proximal section 1680 of the conduit 1620. The righthand illustration shows the balloon 1655 inserted into a distal section 1685 of the conduit 1620, and inflated to expand the distal section 1685 of the conduit 1620. Note that the valves 1610 in FIGS. 16A and 16B are open to allow the balloon 1655 to move through conduit 1620 and the valve 1610.

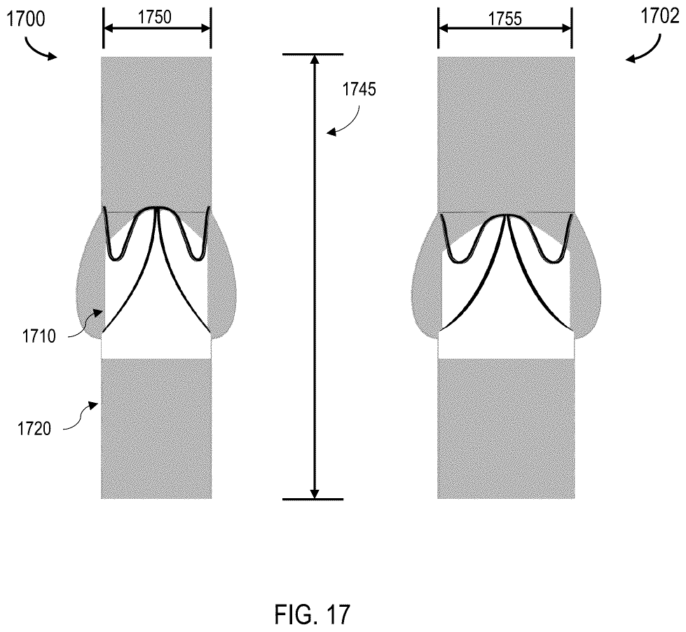

FIG. 17 illustrates a conduit 1720 and valve 1710 before radial expansion and after radial expansion, in accordance with various embodiments. The lefthand side of FIG. 17 shows a pre-expansion example 1700 of a conduit 1720 and valve 1710 before radial expansion. Before expansion, the device including the conduit 1720 and valve 1710 has a length 1745 and a first diameter 1750. The righthand side of FIG. 17 shows a post-expansion example 1702 of the conduit 1720 and valve 1710 after radial expansion. After expansion, the device including the conduit 1720 and valve 1710 has a length 1745 and a second diameter 1755. In particular, after expansion, the conduit 1720 and valve 1710 have about the same length 1745 as before expansion, but the diameter has expanded from a first diameter 1750 to a second diameter 1755. The conduit material is selected such that radial expansion results in minimal to no linear contraction. Properties of the conduit in conjunction with the conduits fixed ends results in minimal contraction of the length during expansion.

According to various implementations, the conduit material may be an expandable synthetic material, and the conduit material may be a polymer, a PTFE, and/or an ePTFE.

In various embodiments, the conduit and valve systems described herein can be non-expandable conduits and/or non-expandable valves.

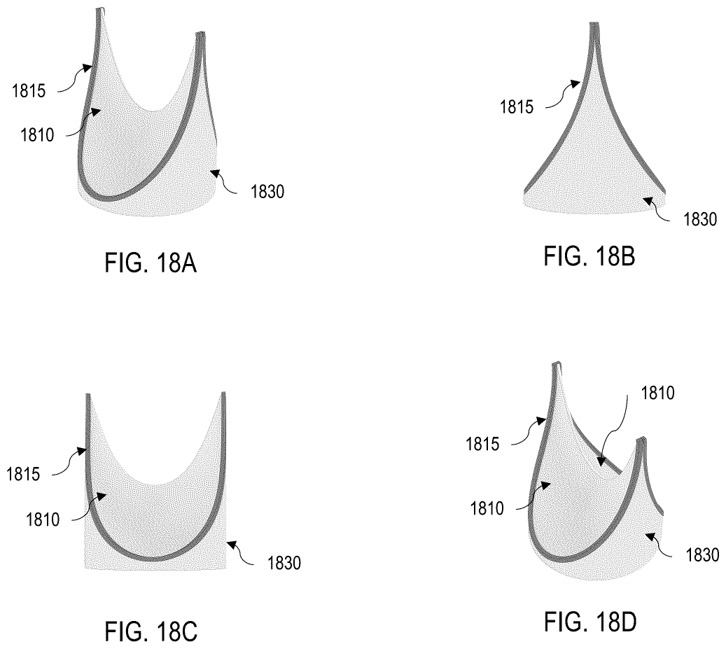

FIGS. 18A-18D illustrate various views of a non-expandable valve device, which can be used in any of the conduit described herein, in accordance with various embodiments. In the embodiment shown, the valve device employs a valve frame 1815 in combination with two valve leaflets 1810 and a sleeve 1830. In particular, FIGS. 18A-18D show a sleeve 1830 coupled to the valve frame 1815. Each of the first and second frame sections may also be described as having a first arm and a second arm denoted by each side of the “U-shape” of the first and second frame sections. In one embodiment, as shown in FIGS. 18A-18D, the valve replacement device includes first and second leaflets 1810, respectively, forming the valve leaflets, attached to the frame 1815. The valve frame 1815 may also include a gap disposed between first and second frame sections of the valve frame 1815, and the sleeve 1830 extends over the gap to impede flow of fluid through the gap. As shown in FIGS. 18A-18D, the sleeve 1830 is configured to align with and attach to the first and second frame sections of the valve frame 1815.

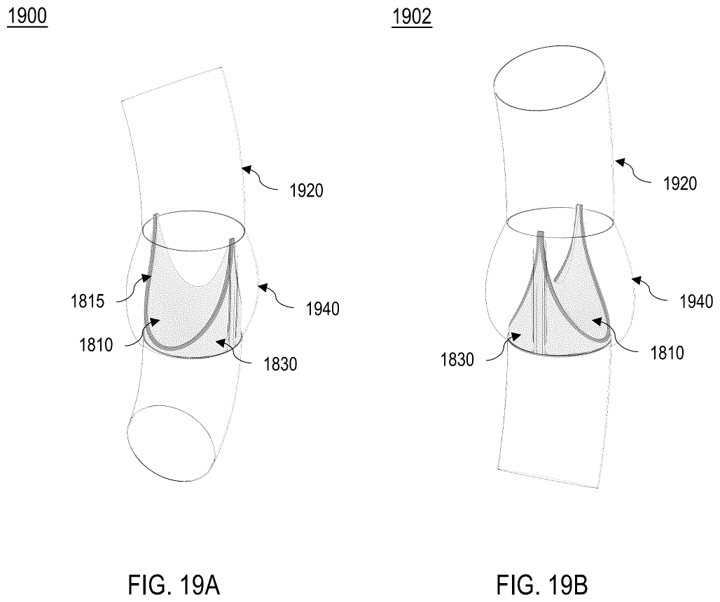

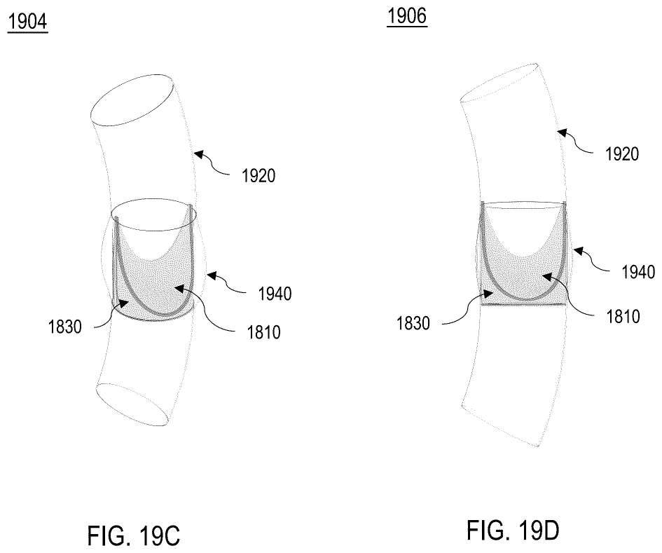

FIGS. 19A-19D illustrate various views 1900, 1902, 1904, 1906 of a non-expandable conduit including a non-expandable valve, in accordance with various embodiments. The non-expandable valve of FIGS. 19A-19D may be the valve discussed above with reference to FIGS. 18A-18D. The non-expandable conduit and non-expandable valve of FIGS. 19A-19D also includes one or more sinuses 1940 as described herein. In particular, the one or more sinuses 1940 are located behind the valve leaflets 1810. In some examples, the area of the conduit 1920 in which the one or more sinuses 1940 are located can have a larger perimeter than the perimeter of the conduit 1920 above and below the valve. In some examples, the area of the conduit 1920 in which the one or more sinuses 1940 are located can have a variable perimeter that varies from proximal end of the sinus 1940 to a distal end of the sinus 1940. The proximal end of the sinus 1940 can be located at a portion of the conduit 1920 that aligns with the proximal end of the valve. The distal end of the sinus 1940 can be located at a portion of the conduit 1920 that aligns with the distal end of the valve. The perimeter of the conduit 1920 at the area where the sinuses 1940 are located, and thus in the area of the valve, is larger than the perimeter of the conduit 1920 above and below the valve.

### SELECT EXAMPLES

Example 1 provides a device, including a flexible conduit, where the conduit is configured to be radially expandable to increase a conduit perimeter while maintaining a consistent conduit length; and an expandable valve configured to be positioned within the flexible conduit, the expandable valve including a frame defining a valve opening, the frame configured to be radially expandable to increase a valve opening size, at least one leaflet coupled to the frame, the leaflet having an open position and a closed position, where in the closed position, the leaflet at least partially covers the valve opening, and an attachment area, coupled to the frame and configured to prevent flow around the valve.

Example 2 provides the device of any of the examples above and/or below, where the flexible conduit includes at least one sinus, the sinus aligned with the at least one leaflet, where the sinus is a bulbous section of the conduit having a sinus perimeter, and where the sinus perimeter is larger than the conduit perimeter.

Example 3 provides the device of any of the examples above and/or below, where the sinus perimeter varies along a length of the sinus from a bottom of the expandable valve to a top of the expandable valve.

Example 4 provides the device of any of the examples above and/or below, where the at least one leaflet includes a plurality of leaflets, the plurality of leaflets having the open position and the closed position, and where in the closed position, the plurality of leaflets close the valve opening.

Example 5 provides the device of any of the examples above and/or below, where the sleeve is attached to the flexible conduit.

Example 6 provides the device of any of the examples above and/or below, where a bottom perimeter of the sleeve at a base of the expandable valve is attached to the flexible conduit.

Example 7 provides the device of any of the examples above and/or below, where a top portion of the expandable valve remains unattached to the flexible conduit.

Example 8 provides the device of any of the examples above and/or below, where the flexible conduit includes a support structure having a plurality of longitudinal members and a plurality of radial members.

Example 9 provides the device of any of the examples above and/or below, where the plurality of radial members are flexible members and the plurality of longitudinal members are rigid members.

Example 10 provides the device of any of the examples above and/or below, where the conduit is radially expandable to permit an increase in a conduit diameter from about 5 mm to about 50 mm.

Example 11 provides a device, including a conduit; and a valve configured to be positioned within the conduit, the valve including a frame defining a valve opening, at least one leaflet coupled to the frame, the leaflet having an open position and a closed position, where in the closed position, the leaflet at least partially covers the valve opening, and an attachment area, coupled to the frame and configured to prevent flow around the valve; where the conduit includes at least one sinus between a proximal section of the conduit and a distal section of the conduit, the sinus aligned with the at least one leaflet, where the sinus is a bulbous section of the conduit having a sinus perimeter, and where the sinus perimeter is larger than a conduit perimeter at the proximal section of the conduit.

Example 12 provides the device of any of the examples above and/or below, where the sinus perimeter varies along a length of the sinus from a bottom of the expandable valve to a top of the expandable valve.

Example 13 provides the device of any of the examples above and/or below, where the sinus perimeter is larger at a proximal portion of the sinus than at a distal portion of the sinus.

Example 14 provides the device of any of the examples above and/or below, where the sinus has a sinus length between the proximal section of the conduit and the distal section of the conduit, and where the sinus perimeter increases to a maximum sinus perimeter about halfway along the sinus length.

Example 15 provides the device of any of the examples above and/or below, where the at least one leaflet includes a plurality of leaflets, the plurality of leaflets having the open position and the closed position, and where in the closed position, the plurality of leaflets close the valve opening.

Example 16 provides the device of any of the examples above and/or below, where a bottom perimeter of the sleeve at a base of the expandable valve is attached to the conduit.

Example 17 provides the device of any of the examples above and/or below, where a top portion of the expandable valve remains unattached to the flexible conduit.

Example 18 provides a device comprising: a conduit having a proximal section, a middle section, and a distal section; a valve configured to be positioned within the conduit, the valve having an open position and a closed position, wherein in the closed position, a valve opening is closed; and an attachment area configured to prevent flow around the valve; wherein the conduit includes at least one sinus in the middle section of the conduit, the sinus aligned with the valve, wherein the sinus is a bulbous section of the conduit having a sinus perimeter, and wherein the sinus perimeter is larger than a conduit perimeter at the proximal section of the conduit.

Example 19 provides the device of any of the examples above and/or below, wherein the sinus perimeter varies along a length of the sinus from a bottom of the expandable valve to a top of the expandable valve.

Example 20 provides the device of any of the examples above and/or below, wherein the sinus perimeter is larger at a proximal portion of the sinus than at a distal portion of the sinus.

Example 21 provides the device according to any of the examples above and/or below, wherein the attachment area is a sleeve.

Example 22 provides the device according to any of the examples above and/or below, examples wherein the attachment area is a suture line.

Example 23 provides the device according to any of the examples above and/or below, wherein the at least one sinus is an expandable sinus, wherein the flexible conduit is expandable from a first conduit perimeter to a second conduit perimeter, the second conduit perimeter larger than the first conduit perimeter, wherein the sinus perimeter is expandable from a first sinus perimeter to a second sinus perimeter, the second sinus perimeter larger than the first sinus perimeter, and wherein the first sinus perimeter is larger than the first conduit perimeter and the second sinus perimeter is larger than the second conduit perimeter.

Example 24 provides the device according to any of the examples above and/or below, wherein the flexible conduit includes a support structure surrounding the conduit.

Example 25 provides the device according to any of the examples above and/or below, wherein the support structure includes a plurality of longitudinal members and a plurality of radial members.

Example 26 provides the device according to any of the examples above and/or below, wherein the plurality of radial members are flexible members and the plurality of longitudinal members are rigid members.

Example 27 provides the device according to any of the examples above and/or below, wherein the support structure includes a mesh.

Example 28 provides the device according to any of the examples above and/or below, further comprising a support structure surrounding the conduit.

Example 29 provides the device according to any of the examples above and/or below, further comprising a first support structure surrounding the proximal section of the conduit and a second support structure surrounding the distal section of the conduit.

Example 30 provides the device according to any of the examples above and/or below, further comprising a third support structure in the middle section of the conduit.

Example 31 provides the device according to any of the examples above and/or below, wherein the support structure includes a gap in the middle section of the conduit around the at least one sinus.

Example 32 provides the device according to any of the examples above and/or below, wherein the conduit is radially expandable to permit an increase in a conduit diameter from about 2 mm to about 20 mm.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize. These modifications may be made to the disclosure in light of the above detailed description.

What is claimed is:

1. A device, comprising:
a flexible conduit, wherein the conduit is configured to be radially expandable to increase a conduit perimeter while maintaining a consistent conduit length; and
an expandable valve configured to be positioned within the flexible conduit, the expandable valve comprising:
a frame defining a valve opening, the frame configured to be radially expandable to increase a valve opening size,
at least one leaflet coupled to the frame, the leaflet having an open position and a closed position, wherein in the closed position, the leaflet at least partially covers the valve opening, and
an attachment area, coupled to the frame and configured to prevent flow around the valve.

2. The device of claim 1, wherein the flexible conduit includes at least one sinus, the sinus aligned with the at least one leaflet, wherein the sinus is a bulbous section of the conduit having a sinus perimeter, and wherein the sinus perimeter is larger than the conduit perimeter.

3. The device of claim 2, wherein the at least one sinus is an expandable sinus,
wherein the flexible conduit is expandable from a first conduit perimeter to a second conduit perimeter, the second conduit perimeter larger than the first conduit perimeter,
wherein the sinus perimeter is expandable from a first sinus perimeter to a second sinus perimeter, the second sinus perimeter larger than the first sinus perimeter, and
wherein the first sinus perimeter is larger than the first conduit perimeter and the second sinus perimeter is larger than the second conduit perimeter.

4. The device of claim 2, wherein the sinus perimeter varies along a length of the sinus from a bottom of the expandable valve to a top of the expandable valve.

5. The device of claim 1, wherein the at least one leaflet includes a plurality of leaflets, the plurality of leaflets having the open position and the closed position, and wherein in the closed position, the plurality of leaflets close the valve opening.

6. The device of claim 1, wherein a bottom perimeter of the attachment area at a base of the expandable valve is attached to the flexible conduit.

7. The device of claim 6, wherein a top portion of the expandable valve remains unattached to the flexible conduit.

8. The device of claim 1, wherein the flexible conduit includes a support structure surrounding the conduit.

9. The device of claim 8, wherein the support structure includes a plurality of longitudinal members and a plurality of radial members.

10. The device of claim 9, wherein the plurality of radial members are flexible members and the plurality of longitudinal members are rigid members.

11. The device of claim 8, wherein the support structure includes a mesh.

12. A device, comprising:
a conduit; and
a valve configured to be positioned within the conduit, the valve comprising:
a frame defining a valve opening,
at least one leaflet coupled to the frame, the leaflet having an open position and a closed position, wherein in the closed position, the leaflet at least partially covers the valve opening, and
an attachment area, coupled to the frame and configured to prevent flow around the valve;
wherein the conduit includes at least one sinus between a proximal section of the conduit and a distal section of the conduit, the sinus aligned with the at least one leaflet, wherein the sinus is a bulbous section of the conduit having a sinus perimeter, and wherein the sinus perimeter is larger than a conduit perimeter at the proximal section of the conduit.

13. The device of claim 12, wherein the sinus perimeter varies along a length of the sinus from a bottom of the valve to a top of the valve.

14. The device of claim 13, wherein the sinus perimeter is larger at a proximal portion of the sinus than at a distal portion of the sinus.

15. The device of claim 12, wherein the sinus has a sinus length between the proximal section of the conduit and the distal section of the conduit, and wherein the sinus perimeter increases to a maximum sinus perimeter about midway along the sinus length.

16. The device of claim 12, wherein a bottom perimeter of the attachment area at a base of the valve is attached to the conduit.

17. The device of claim 16, wherein a top portion of the valve remains unattached to the conduit.

18. The device of claim 12, further comprising a support structure surrounding the conduit.

19. The device of claim 12, further comprising a first support structure surrounding the proximal section of the conduit and a second support structure surrounding the distal section of the conduit.

20. A device, comprising:
a conduit having a proximal section, a middle section, and a distal section;
a valve configured to be positioned within the conduit, the valve having an open position and a closed position, wherein in the closed position, a valve opening is closed; and
an attachment area between the valve and the conduit configured to couple the valve to the conduit and prevent flow around the valve;
wherein the conduit includes at least one sinus in the middle section of the conduit, the sinus aligned with the valve, wherein the sinus is a bulbous section of the conduit having a sinus perimeter, and wherein the sinus perimeter is larger than a conduit perimeter at the proximal section of the conduit.
